(19) United States
(12) Patent Application Publication
(10) Pub. No.: US 2008/0112902 A1
(43) Pub. Date: May 15, 2008

(54) Protective Dental Balm and Method

(76) Inventor: Joseph Perechocky, Highland Mills, NY (US)

Correspondence Address: CALHELHA & DOYLE LLC, 2640 ROUTE 9W, CORNWALL, NY, 12518, US

(21) Appl. No.: 11/940,318

(22) Filed: Nov. 14, 2007

Related U.S. Application Data
(63) Continuation-in-part of application No. 11/113,855, filed on Apr. 25, 2005, now abandoned.

(51) Int. Cl. A61K 8/21

(52) U.S. Cl. 424/52

(57) ABSTRACT

A dental balm composition and method prevents damage to teeth which are prone to prolonged exposure to air or other drying conditions and which are therefore susceptible to tooth decay. The dental balm composition and uses an active decay preventing agent such as fluoride ion, xylitol or glycerin dispersed in a water insoluble adhesive and protective balm base such as petrolatum along with optional thickeners, flavorants and the like. The method of damage prevention applies the balm, such as by swab or finger, to the exposed surfaces of the subject's front teeth prior to vigorous physical activity, exercise, prolonged episodes of mouth breathing, or other harsh or deleterious conditions including drying conditions during strenuous periods of exercise or tooth whitening procedures.

### FIELD OF THE INVENTION

The present invention relates to a dental balm composition and a method of using a dental balm to prevent or minimize damage to teeth, particularly caused when teeth are permitted to become dry, and to reduce sensitivity due to dehydration of teeth after tooth whitening procedures. The invention provides a composition and method designed to prevent or minimize dental decay in athletes and others prone to dry mouth conditions, and to minimize dental sensitivity immediately after tooth whitening procedures, whether at home or a dental office.

### BACKGROUND OF THE INVENTION

Many people suffer from dry mouth conditions from time to time, but this can present a serious problem for individuals who suffer from dry mouth as a chronic condition. Dry mouth presents itself in a variety of circumstances and causes dental problems when the surfaces of teeth become dry as by overexposure to air. It is well known that antibodies in saliva are useful in the prevention of cavities or dental caries, however, the beneficial effects of saliva are not possible when, for whatever reason, teeth are permitted to become dry

Athletes, particularly those involved in aerobic sports, are prone to dry mouth conditions. It is recognized that aerobic athletes spend a great deal of time breathing through their mouths causing tooth enamel surfaces to dry out, and increasing the prevalence of dental caries in these individuals, especially on their front teeth. Athletes subject to this condition would include runners, swimmers, bicyclists, skiers and long-distance competitors of all types.

Other persons who may be prone to dry mouth and teeth include individuals who, for various reasons, breathe principally through their mouths. Individuals with dental or skeletal malocclusion with anterior open bite (i.e. the front teeth do not meet, leaving space between upper and lower teeth) sometimes experience problems with improperly closing lips. Other individuals prone to dry mouth and teeth include those with sinus and related breathing problems, as well as other persons who suffer from excessive snoring. All of these persons may develop damage to their tooth enamel due to the inaction of saliva when their mouth and teeth become dry.

Others who may benefit from the composition and method of the present invention are persons with xerostomia having compromised or non-functioning salivary glands (such as certain radiation therapy patients and people with Sjogren's syndrome, diabetes and vitamin deficiencies) as well as persons taking certain medications including some anti-depressives, anti-hypertensives, diuretics and anti-histamines.

Thirsty runners consume sports drinks and juices, many with citric acid, and these drinks are also known to increase tooth decay. It is believed that the present composition may benefit infants prone to nursing bottle mouth, but this has not been evaluated to date. Applying the dental balm on the teeth at a time when the fluoride would do the most good, such as right before athletic activity, before going to bed, and immediately after tooth whitening is a preferred method of practice. For example, asthma oral inhalers when sprayed into the mouth tend to dry the mouth and leave a sticky film on the teeth. Applying the protective balm to the teeth before spraying the inhaler into the mouth will protect the teeth.

In the mouth, teeth are naturally protected from decay by saliva. When saliva is removed from the front teeth, as when an athlete is involved in long-distance running or swimming with the mouth open, or where a person is mouth breathing throughout the night while sleeping, tooth enamel is more susceptible to destruction by naturally occurring acids in the mouth produced by Streptococcus Mutans. S. Mutans is a bacteria that is always present in the mouth and in the presence of carbohydrates produces acids that de-mineralize tooth enamel and cause cavities. This is especially true for cervical and root cavities when some of the tooth's root is exposed. Additionally, with decreased saliva there is an increase in bacteria which causes an increase in plaque formation. Traditional fluoride treatments help to prevent cavities by re-mineralizing tooth enamel when the fluoride is incorporated into the calcium phosphate matrix of the enamel.

In general, traditional cavity preventing formulations, particularly tooth pastes, whether used separately or in combination, are known in the art.

Consumers are most familiar with oral hygiene products delivered in the form of toothpastes but balm and ointment treatments are also known. Dental compositions for the treatment of hypersensitive teeth are known and may be applied to teeth in balm form and may be applied via fingertip. U.S. Pat. No. 6,761,879 discloses the use of titanium compounds to prevent dental caries and specifies that these may be used in balm form. A further U.S. Patent Application Publication 2002/0137728 discloses topical oral care compositions using ascorbyl phosphate which may be delivered to the oral cavity by means of a tooth balm.

The present invention overcomes the problems presented by dry teeth by providing a decay preventative material in a dental balm composition capable of both delivering an active ingredient to the surface of teeth and maintaining it there, while simultaneously minimizing the exposure of tooth surfaces to the drying effect of exposure to harsh conditions including dry air, or dehydration due to whitening procedures. Enamel dehydration also causes sensitivity immediately after tooth whitening and other dental and periodontal procedures.

The dental balm composition used in the present invention may be readily applied to the subject's teeth, with a swab or forefinger, prior to sleep, athletic activity or other activities which contribute to dry mouth conditions and applied to the teeth immediately after tooth whitening procedures.

The advantage of the present invention over the known art is provided by the combination of an active decay preventing material and a water insoluble protectant adhesive dental balm base material which is capable of delivering and maintaining the active ingredient in contact with the surface of the teeth during periods of strenuous breathing or other drying conditions.

A general objective of the invention is to promote dental health in persons subject to strenuous activity and breathing conditions.

Another broad objective of the invention encompasses a dental balm composition and method for the prevention of damage caused to dry tooth surfaces upon prolonged exposure to air.

A more specific object of the invention is to provide a fluoride tooth balm composition to prevent or reduce the incidence of caries on front teeth in long-distance athletes such as runners or swimmers and other people who breathe primarily through their mouths.

A further object is to provide a dental balm for reducing sensitivity in teeth undergoing whitening procedures, due to dehydration of the enamel from the procedure.

In the method of the present invention, damage caused by prolonged exposure of dry teeth to air is minimized by application of a dental balm comprised of an water insoluble protectant adhesive material in combination with an active decay preventative material, applying the balm to the surface of the exposed teeth and maintaining the active ingredient in close proximity to the surface of teeth during periods of strenuous activity, mouth breathing or sensitivity due to whitening procedures.

These objects, as well as others which will be apparent, are achieved generally by providing a dental balm composition and method of use for the prevention of damage via dehydration to teeth which are subject to prolonged exposure to air, as by vigorous aerobic exercise, a variety of breathing difficulties including excessive mouth breathing and sensitivity from whitening procedures.

### SUMMARY OF THE INVENTION

The dental balm composition is comprised of a water insoluble protective adhesive base material and a decay preventative agent in an amount effective for protecting teeth during strenuous activities, exposure to air, and other drying conditions. The protective adhesive base material is effective for delivering the decay preventive agent to the required dental site and maintaining it there for the duration of the subject activity or exposure.

The water insoluble protective adhesive base material is preferably petrolatum which is a semi-solid mixture of hydrocarbons obtained from petroleum. Petrolatum is also known as petroleum jelly and is commonly used as a lubricant or medicinal ointment.

The water insoluble protective adhesive base material may additionally contain a thickening agent to adjust the consistency of the desired final balm composition. The thickening agent may be flour, talc, arabic gum and other gum additives, starch, silica, beeswax and other waxes or other colloidal stabilizer.

The dental balm composition of the present invention contains an active decay preventive or minimizing agent such as fluoride ion, xylitol or glycerin.

The fluoride ions are generally obtained by utilizing sodium fluoride or stannous fluoride compounds approved for dental treatments. Particularly preferred are fluoride treatments in aqueous solutions (such as rinses) which may be readily mixed or emulsified in the water insoluble protective adhesive base material.

The balm formulation and method work directly on dry teeth particularly when an active ingredient is emulsified in an adhesive balm which sticks to the teeth and maintains the active agent in close proximity. When fluoride is the active agent, it may contain the same concentration of fluoride that is present in saliva immediately after rinsing with a fluoride mouth rinse, and it maintains the agent on the teeth until it is brushed off. Other active agents are xylitol or glycerin. Although the balm itself may act as a barrier, maintaining the agent near the tooth surfaces is a very beneficial advantage of the subject method.

The dental balm composition described above is used in the method of the present invention to prevent damage, such as tooth decay, experienced when teeth are permitted to become dry. The dental balm of the present invention is easily swabbed, via finger or applicator, onto the frontal aspect (front surfaces) of the upper and lower teeth prior to overexposure to air or drying conditions, or to sensitive teeth after exposure to whitening procedures. The adhesive quality of the balm base enables it to remain in close proximity to the areas of the tooth surfaces most affected by exposure and delivers the active decay preventive agent, such as fluoride, where it is most useful. It is preferred that the dental balm composition be applied to clean teeth and removed after use by brushing with toothpaste.

Other objects, features and advantages of the present invention will be apparent upon consideration of the following detailed description of the invention and the examples, which should be construed in an illustrative and not limiting sense.

### BRIEF DESCRIPTION OF THE DRAWINGS

FIG. 1 is a cross-section representation of a tooth treated with a protective balm.

FIG. 2 is a schematic representation of a protective dental balm with active and inactive components.

FIG. 3 is a schematic representation of an embodiment of a protective dental balm treatment.

FIG. 4 is process diagram for a method of the present invention.

### DETAILED DESCRIPTION OF THE INVENTION

FIG. 1 represents a dental balm treatment system 1 where a tooth structure 2, having outer surfaces 3, is treated with protective dental balm 4. The tooth structure may be enamel, dentine, cementum or related dental structures. In this embodiment, tooth structure 2 is shown in a typical cross-section and has been treated with a portion of protective dental balm 4 which has been applied to an outer surface of a tooth or tooth structure. Preferably the teeth have been cleaned, as by brushing and flossing, before application of the protective dental balm. The protective balm may be applied in a variety of ways including use of an applicator, a swab or smeared with the user's finger.

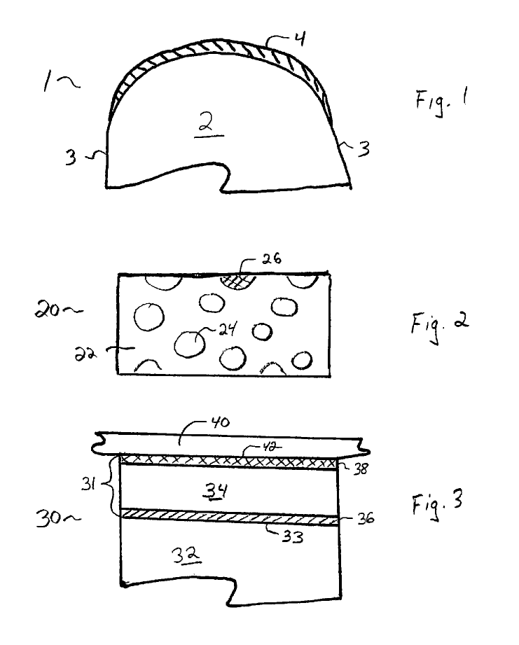

FIG. 2 is a schematic representation of a cross-section of protective dental balm 20 and its principle components. In this embodiment, adhesive base 22, such as petrolatum or beeswax and mixtures, contains active and inactive ingredients, such as in this embodiment protective agent 24, such as fluoride, xylitol or glycerin, and flavor agent 26, such as peppermint. These ingredients can be mixed in a variety of ways but will preferably be emulsified, as discussed, below, for consistency and effectiveness.

FIG. 3 is a schematic representation of a dental protection treatment system 30. In this embodiment, a portion of tooth structure 32 having outer surface 33 has been treated with an application of protective dental balm 31. In this figure, balm 31 is depicted for clarity as comprising adhesive base material 34, active protective ingredient 36 such as fluoride, xylitol or glycerin, nearest the dental surface 33. Also represented in this figure is a portion of a user's tongue 40 having a surface 42 which may be near or touching protective balm 31 and particularly a pleasant flavoring agent 38.

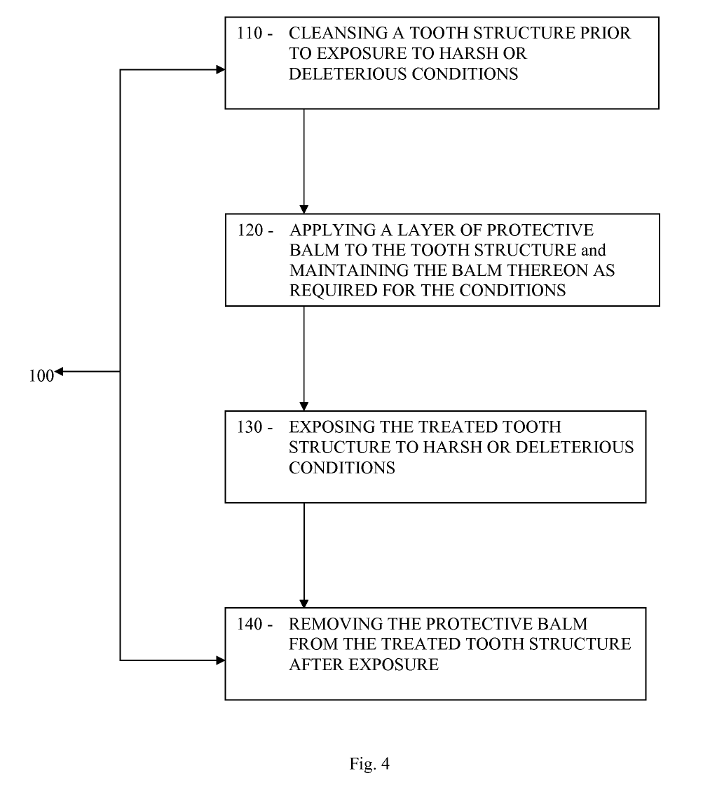

FIG. 4 is a process flow chart for a preferred embodiment of the present invention. A protective dental balm treatment system 100 and method of practicing the same is provided. The method and system has an initial cleaning step 110 wherein a tooth structure is cleansed before exposure to harsh or deleterious conditions, including drying or whitening conditions as discussed above. Cleaning of the tooth structure, preferably cleaning all of the teeth to be treated, typically is accomplished by brushing with toothpaste, as well as optionally flossing, as may be required or desired by the user. Thereafter, a layer of protective dental balm, having a formulation as described in the examples, is applied to the tooth structure in step 120. The preferred dental balm compositions have at least one active ingredient, such as xylitol, glycerin or the various fluoride compounds discussed above, which are know to have varying degrees of cavity minimizing or prevention properties. The active ingredient is delivered by an adhesive base material, such as petrolatum, waxes, mixtures of these and the like, whereby the adhesive base effectively maintains such active ingredients upon or in close proximity to the surface of a tooth structure.

A tooth structure treated with a protective balm in the aforementioned manner may thereafter be exposed in step 130 to the harsh or deleterious conditions described above which are typically expected to cause damage to otherwise exposed teeth.

In another embodiment of the subject method, applying the protective balm may also includes the additional step of preventing tooth structures from becoming dry or conversely, maintaining moisture including saliva or water on or in proximity to the treated tooth structure. Drying is prevented or minimized when the protective balm coating adheres closely to the treated tooth structure and dental surfaces.

After the teeth have been exposed to a period of the aforementioned harsh or deleterious conditions, and preferably when such conditions have ceased, the protective dental balm may be removed from the treated tooth structure in step 140. Such removal may be accomplished by wiping the treated tooth structure with a clean, dry napkin, towel, swab or similar cloth or paper product. Preferably the subject user of the protective dental balm will once again clean the treated teeth by brushing with toothpaste and flossing, as may be appropriate or desired.

In accordance with the present invention a dental balm composition and method for protecting teeth is provided to promote dental health in persons experiencing deleterious drying effects from harsh conditions including prolonged air exposure or harsh whitening procedures.

The dental balm composition is comprised of a water insoluble protective adhesive base material and a decay minimizing or preventative agent in an amount effective for preventing or minimizing dental caries.

The water insoluble protective adhesive base material is preferably petrolatum (petroleum jelly) which is a semi-solid mixture of hydrocarbons obtained from petroleum. The dental balm composition of the present invention requires a water insoluble protectant adhesive. Commercially available white petrolatum, USP is available for consumer use and is particularly effective for use in the present application. Petrolatum has a specific gravity of about 0.815 to 0.880 at 60° C.

Alternative protective adhesive base materials may include any medically approved water insoluble ointment, gel or balm formulations capable of carrying the active ingredients, and applying and maintaining same to the affected teeth in the prescribed manner. One such alternative protective adhesive base material is wax such as beeswax. Combinations of wax and petrolatum are useful.

The water insoluble protective adhesive base material such as petrolatum may additionally contain a thickening agent to adjust the consistency of the desired final balm composition. The thickening agent may be selected from flour, talc, starch, silica, wax (such as beeswax), gums such as agar-agar, Arabic, locust bean, carrageen, guar, karaya, and tragacath gums or other thickening agents. Some of these may benefit from the use of chemical preservatives as discussed below.

Stabilizers, such as the gums, may be used to prevent degradation of the products, and as protective colloids, thickeners and emulsifying agents as is common in food and pharmaceutical products, including toothpastes.

Thus the viscosity and adhesiveness of the subject dental balm composition may be readily adjusted to ensure effective consumer packaging, ease of removal of the balm from its container, ease of application and effective adhesiveness of the product after application to the consumer's teeth and effective removal from the teeth when the treatment is finished. It will be recognized that all ingredients and additives utilized in the dental balm composition of the present invention are selected in forms approved for oral applications.

Some formulations may include chemical preservatives to retard spoilage of the balm, especially when certain optional compounds prone to spoilage (e.g. flour) are used therein. Common chemical preservatives include benzoates, such as sodium benzoate, nitrites, such as sodium nitrite, sulphites such as sulphur dioxide, and sorbic acid. These and other chemical preservatives are effective for killing or inhibiting the bacteria which cause spoilage.

Dry, solid active agents are less preferred as additives in the water insoluble protective adhesive base material which will tend to cover such particles and hold them away from the teeth. Inactive ingredients such as thickeners or stabilizers may be dry so long as they can be efficiently mixed in the balm composition. Liquid additives which can be homogenously mixed or emulsified can disperse the active ingredients uniformly in the base and in close contact with the treated teeth.

The dental balm composition of the present invention contains an active decay preventive agent which is preferably fluoride ion obtained by utilizing sodium fluoride or stannous fluoride compounds. More preferably, the fluoride ion may be contained in aqueous solution which may optionally also include alcohol. It will be understood that the fluoride concentration in the balm of the present invention can be readily adjusted by manipulation of either the fluoride concentration in the aqueous solution or in the amount of such solution used in the insoluble base material. The fluoride concentration should be sufficient to be effective yet not so great as to be unsafe or separate out of the dental balm composition.

Taste of the dental balm may be improved with small amounts of flavorants or sweeteners. Low calorie additives are preferred because sugar based sweeteners and flavors would be counterproductive to the intended purposes of the dental balm composition and method.

It is contemplated that the balm composition of the present invention will be packaged in glass or plastic jars, metal tins, tubes or plastic tubs with lids, in sizes and amounts convenient for athletes and others. Small tubes containing about 11.5 g or small plastic containers containing about 7.5 g of the product would seem particularly useful.

Topical application of fluoride has long been the decay preventing treatment of choice. Two methods of topical application of fluoride are by use of acidulated phosphate-fluoride (APF) gels in trays, or liquid rinses. Sodium fluoride gels and rinses, and stannous fluoride gels are also available. See, for example, Dental Materials: Properties, & Manipulation, Sixth Edition, R. G. Craig, et al., Published by Mosby-Year Book, Inc., 1996 at p. 30.

A neutral gel-like topical sodium fluoride agent is Nupro, from Ash USA/Dentsply. Nupro is thixotropic and its sodium fluoride is thickened by polyacrylic acid and a gum. Its pH is adjusted to between six and eight to minimize acid etching of restorative materials. Another source of fluoride for topical applications is Thera-Flur®-N brand of neutral sodium fluoride gel-drops which provide 0.5% fluoride ion (F—) from 1.1% (w/v) sodium fluoride (NaF) at neutral pH, and is available by prescription from Colgate Oral Pharmaceuticals, Inc. Its inactive ingredients include purified water, sorbitol, propylene glycol, hydroxyethylcellulose, methyl paraben and propyl paraben. These and other sources of fluoride ion may be combined with the insoluble base material of the present invention but are less preferred than fluoride solutions such as the rinses discussed next.

The fluoride ion active agent used in the present dental balm composition and method is preferably provided in a solution. This offers the advantage of convenient manipulation or adjustment of the fluoride ion concentration for a variety of treatment effects, as necessary. Note that although fluorosis is not generally a problem when fluoride treatments are applied topically, nonetheless, users of the subject dental balm composition should not swallow the product.

The active fluoride agent may be conveniently incorporated in the dental balm composition by utilizing a commercially available consumer fluoride rinse product such as ACT® brand fluoride rinse, from Chattem, Inc., Chattanooga, Tenn. The active ingredient in ACT® brand rinse is sodium fluoride at a level of 0.05%, providing a fluoride ion concentration of approximately 0.02% w/v in the rinse. ACT® brand rinses are preferred for use in compositions of the present invention because they are readily available and are offered in several flavors as well as alcohol free versions. The inactive ingredients in the commercial rinse formulations include water, preservatives, stabilizers, colorants, sweeteners and the like. Additional fluoride rinses are available from Colgate Oral Pharmaceuticals, Inc. Gel-Kam® brand oral care rinse is a 0.63% w/w stannous fluoride concentrate which, when diluted according to directions provides 0.1% w/v stannous fluoride and contains glycerin as an inactive ingredient. Colgate PreviDent® rinse provides 0.2% w/v neutral sodium fluoride in a mint-flavored, neutral, aqueous solution containing 6% alcohol.

Fluoride may be provided by sodium fluoride or stannous fluoride in other forms where their respective compositions are adjusted to achieve suitable concentrations of active fluoride ion in the dental balm product.

For dental balm compositions of the present invention which utilize a fluoride-containing liquid rinse as the active ingredient, a preferred formulation will contain about three parts by volume of the water insoluble adhesive base material such as petrolatum per one part by volume aqueous fluoride-containing rinse. For instance, suitable formulations may combine 15 ml petrolatum with 5 ml of ACT® brand fluoride rinse. Five milliliters of such liquid rinse appears to be about the practical limit unless mixing or emulsifying agents are used. Up to about 7.5 ml can be utilized when effective mixing or emulsifying agents are used. Compositions containing less than about 2 ml of the fluoride-containing rinse would tend to be less efficacious or would begin to require increasing concentrations of fluoride in the aqueous solution. Dental balm compositions made in accordance with these formulations will typically incorporate about one half part by volume of thickening agent to adjust the balm to a desired consistency. Artificial sweetener may be used to taste.

A protective balm formulation substituting xylitol wood sugar (C₅H₁₂O₅) as an active ingredient instead of sodium fluoride can be used. Xylitol may be dissolved in purified water to a concentration of about 15-40 percent and preferably about 20-30 percent. A concentration of about 26 percent has been used. The xylitol solution is then emulsified in an adhesive base, preferably comprised of beeswax and petrolatum. Polysorbates such as Tween 20 or Tween 80 may be readily utilized as emulsifying agents. Food color, peppermint flavor and preservatives may be preferably included.

Analogous formulations utilizing other active ingredients, or combinations of these also contemplated. For example, glycerin may be utilized as a moisturizing agent in the present balm composition and method. Another active agent may be potassium nitrate.

It should be understood that both wet and dry measurements are given in milliliters to denote relative volumes of the respective ingredients. Approximations of weights are given in grams where known or calculated.

As noted, prolonged exposure to vigorous breathing or whitening procedures tends to dry tooth surfaces such that saliva cannot readily perform its natural decay preventive function. The subject dental balm is easily swabbed, via finger or applicator, onto the front surfaces of teeth prior or after such exposure. The adhesive quality of the balm base enables it to remain in close proximity to the areas of the tooth surfaces most affected and delivers the active decay preventive agent, such as fluoride, where it is most useful.

In the preferred method of the present invention, the teeth will be cleaned by the consumer, as by brushing and flossing, prior to application of the dental balm. Brushing with toothpaste is particularly preferred. The adhesive dental balm is then applied to protect the teeth prior to athletic activity or other exposure of the teeth, or to sooth sensitive teeth immediately after whitening procedures, after which the dental balm composition is readily removed, again preferably by brushing and flossing.

### EXAMPLE 1

A dental balm composition in accordance with the present invention was prepared by combining in a glass bowl one tablespoon (approximately 15 ml) of white petrolatum, USP with 1 teaspoon (approximately 5 ml) of cinnamon flavored ACT® brand fluoride rinse. The ingredients were mixed vigorously by hand, whereupon one half teaspoon (approximately 2.5 ml) of talc (Caldesene Protective Powder containing 81% talc and 15% zinc oxide) was added to the composition. Mixing continued until all ingredients were fully incorporated into a pink colored homogenous dental balm composition. A similar formulation utilizing approximately 2 teaspoons fluoride rinse did not mix satisfactorily by hand but would benefit from mechanical mixing or use of a mixing aid or emulsifier.

### EXAMPLE 2

A second dental balm composition in accordance with the present invention was prepared by combining in a glass bowl one tablespoon (approximately 15 ml) of white petrolatum, USP with 1 teaspoon (approximately 5 ml) of mint flavored ACT® brand fluoride rinse. The ingredients were mixed vigorously, whereupon one half teaspoon (approximately 2.5 ml) of talc (Caldesene Protective Powder) was added to the composition. Mixing continued until all ingredients were fully incorporated into a light green colored homogenous mixture. Samples of the compositions provided in Examples 1 and 2 showed no discernible separation or spoilage of ingredients four months after formulation.

### EXAMPLE 3

Two laboratory specimens of denture shaped typodont artificial dental enamel were rinsed with tap water and placed wet side by side on a lab bench. A portion of the dental balm composition made in accordance with example 1 above was applied to one rinsed specimen and placed back on the lab bench. A small fan directed room temperature air on the treated and untreated specimens for a period of five minutes. At the end of the five minute period each specimen was examined. The untreated specimen, which had only been rinsed with water was dry to the touch after five minutes exposure to the fan. The treated specimen having the dental balm composition applied to the surface remained protected from exposure to the fan air.

### EXAMPLE 4

The procedure of Example 3 was repeated by replacing the tap water rinse with Salivart Synthetic Saliva from Gebauer (75 g can with nitrogen propellant). The specimens were sprayed, treated with the dental balm and exposed to fan air for five minutes. The treated surfaces remained protected after exposure to the fan air.

### EXAMPLE 5

Another dental balm composition was prepared by combining in a glass bowl one tablespoon (approximately 15 ml) of white petrolatum, USP with 1 teaspoon (approximately 5 ml) of cinnamon flavored ACT® brand fluoride rinse. The ingredients were mixed vigorously, whereupon one half teaspoon (approximately 2.5 ml) of Gold Medal brand All Purpose Flour was added to the composition, along with one drop (approximately 0.65 ml) of Sweetleaf Stevia Clear brand artificial sweetener. Mixing continued until all ingredients were fully incorporated into a homogenous mixture. A subject applying this formulation to his teeth found satisfactory performance and taste. After two months, the formulation of this example showed signs of spoilage and would have benefited from the addition of a preservative such as sodium benzoate.

### EXAMPLE 6

The method of the present invention was practiced by an individual, who tested the dental balm composition of example 1 above. First, the subject brushed and flossed his teeth in accordance with a preferred embodiment of the method. Thereupon, the subject placed a portion of the dental balm composition on his forefinger and applied it to the outer surface of his upper teeth by swiping his coated forefinger with the composition on the front surfaces of his upper teeth from approximately behind his left canine across the front upper arch to approximately behind the right canine. The lower arch was left untreated. After an aerobic exercise session of 15 minutes on a Schwinn AirDyne stationary bicycle, the subject inspected his teeth. The lower untreated teeth were relatively dry to the touch, whereas the treated upper teeth retained the dental composition on their surfaces. A second exercise session of a further 15 minutes was undertaken for a total aerobic workout of 30 minutes whereupon the teeth were examined again. The lower untreated teeth were dry to the touch whereas the upper teeth retained the dental balm treatment intact. Light brushing with conventional toothpaste removed the dental balm from the subject's teeth.

### EXAMPLE 7

A dental patient underwent a whitening treatment on his front teeth utilizing Discus Dental Nite White whitening gel in a custom tray for one hour. The active ingredient in the whitening gel was 16% by volume carbamide peroxide. After one hour of treatment, the whitened teeth experienced sensitivity to air and temperature changes due to dehydration of the enamel. The teeth were then treated for 10 minutes with a finger swab of the dental balm composition of Example 1. The teeth were then brushed with fluoride toothpaste whereupon the aforementioned sensitivity was noticeably lessened.

### EXAMPLE 8

A protective balm designated Formulation “A” was made by compounding about 0.01% sodium with the following ingredients. The formulation provided a fluoride ion concentration of about 0.004%, approximating the concentration of fluoride found in saliva immediately after rinsing with a fluoride mouth rinse such as Act or Fluoriguard brand rinses. The fluoride was emulsified in the petrolatum-wax adhesive base, to provide a constant level of fluoride protection to the teeth. The balm contained:

| | | |
|---|---|---|
| Act Fluoride Rinse (mint) | 12.5 | ml |
| Sodium Benzoate | 0.9 | gm |
| Beeswax | 6.0 | gm |
| Petrolatum | 36.6 | gm |
| Tween 20 (emulsifier) | 1.2 | ml |

The Act brand fluoride rinse which was used as the source of the fluoride, had a nominal 0.05% sodium fluoride concentration, providing a fluoride ion concentration of about 0.02%. The emulsifier was polysorbate 20. The dental balm can be made in any color and any flavor, this formulation was peppermint.

### EXAMPLE 9

Ten patients were selected to participate in a preliminary trial study of a dental balm composition having Formulation A, designed to reduce dental decay caused when teeth are permitted to become dry, such as in athletes and others prone to dry mouth conditions, and to reduce sensitivity due to dehydration of teeth immediately after tooth whitening procedures. The patients were instructed to use the protective balm in the above described method. Patient One had Sjogren's Syndrome, complained of dry mouth and was prone to caries. After her dental work was completed, she was instructed to use the dental balm at bed time. At her 6 month fellow up visit, the patient had a decrease in caries and reported a decrease of dry mouth in the morning. Patient Two was a “serious runner”, logging about 25 miles per week, and was prone to caries on his front teeth. His complaint was that there is little saliva in his mouth during a serious run of 6 miles. After using the dental balm four times during his runs, he reported a “refreshing effect on his teeth and mouth.” After continued use, a six month follow up visit revealed fewer caries on his front teeth. Five other patients complained of sensitivity to cold from various sources. After using the dental balm, three patients reported a decrease in sensitivity and two did not. Three other patients used the tooth balm directly after tooth whitening procedures. Two were office procedures and the other was home custom-tray procedure. All three patients reported a decrease in sensitivity approximately one hour after applying the tooth balm.

### EXAMPLE 10

Twenty four patients were selected for a further study involving the efficacy of Formulation A in reduction of tooth sensitivity and alleviation of dry mouth symptoms. The patients were divided randomly into two groups of twelve. One group used the balm containing fluoride as an active ingredient and the other group received a placebo. Both groups were instructed to apply the material to the root surfaces following normal oral hygiene procedures and were told to allow the material to remain on the root surfaces all night without rinsing it away. All patients in the active group reported feeling a noticeable lessening of dryness. Five patients in the placebo group noticed less dryness and the remaining seven reported no difference. Nine patients in the active group reported a significant decrease in tooth sensitivity following periodontal surgery after 4 weeks post-op. The other 3 reported slight decrease only. Only 4 patients in the placebo group reported significant decrease in tooth sensitivity after 4 weeks. The other 8 reported. slight or no change.

### EXAMPLE 11

Another protective balm formulation was made substituting xylitol as an active ingredient instead of sodium fluoride. Xylitol weighing 7.8 g was dissolved in purified water to a concentration of 26 percent. Twenty five ml of the xylitol solution was emulsified with 2.4 ml polysobate 20 (Tween 20) in an adhesive base comprised of 6 g beeswax and 36.6 g petrolatum. Food color, peppermint flavor and 0.9 g sodium benzoate were also included.

The invention now being fully described, it will be apparent to one of ordinary skill in the art that many changes and modifications can be made thereto without departing from the spirit or scope of the invention as set forth herein.

What is claimed is:

1. A method for protecting teeth from damage due to prolonged exposure to harsh and deleterious conditions including the drying effects of air, comprising the steps of:
applying a protective dental balm composition to affected teeth prior to exposure wherein said dental balm composition comprises a water insoluble protective adhesive base material and an effective amount of an active agent for minimizing dental caries; and exposing such treated teeth to conditions expected to cause damage to unprotected tooth surfaces.

2. A method for protecting teeth as in claim 1 further comprising the step of cleansing the subject teeth prior to application of the dental balm composition.

3. A method for protecting teeth as in claim 1 further comprising the step of cleaning the treated teeth after the danger of such exposure has passed by removing the protective dental balm.

4. A method for protecting teeth as in claim 1, wherein said water insoluble protective adhesive base material is selected from the group consisting of petrolatum and wax.

5. A method for protecting teeth as in claim 4, wherein said wax is beeswax.

6. A method for protecting teeth as in claim 1, wherein said water insoluble protective adhesive base material further comprises a thickening agent.

7. A method for protecting teeth as in claim 6, wherein said thickening agent is selected from the group consisting of flour, talc, gum, starch, silica and wax.

8. A method for protecting teeth as in claim 1, wherein said active agent for minimizing dental caries is xylitol.

9. A method for protecting teeth as in claim 1, wherein said active agent for minimizing dental caries is glycerin.

10. A method for protecting teeth as in claim 1, wherein said active decay preventive agent is fluoride ion.

11. A method for protecting teeth as in claim 1, wherein said fluoride ion is provided by sodium fluoride or stannous fluoride.

12. A method for protecting teeth as in claim 1, wherein said fluoride ion is provided in an aqueous solution.

13. A method for protecting teeth as in claim 1, comprising approximately 3 parts by volume water insoluble protective adhesive base material per approximately 0.5-1.5 parts by volume fluoride-containing aqueous solution.

14. A method for protecting teeth as in claim 12, comprising approximately 3 parts by volume water insoluble protective adhesive base material per approximately one part by volume fluoride-containing aqueous solution.

15. A method for protecting teeth as in claim 12, wherein said fluoride-containing aqueous solution contains approximately 0.01 to 0.03 weight percent fluoride ion.

16. A method for protecting teeth as in claim 1, wherein said active agent is potassium nitrate.

17. A method protecting tooth surfaces from damaging conditions, comprising the steps of:
cleansing a tooth structure before exposure to harsh or deleterious conditions; applying a layer of protective dental balm to the exposed surface of a tooth structure; exposing the tooth structure treated with the protective balm to said harsh or deleterious conditions expected to cause damage to otherwise exposed teeth; and removing the protective balm from the treat tooth structure after such exposure.

18. A method as in claim 17 wherein said protective dental balm contains at least one active ingredient selected from the group comprising xylitol, glycerin or fluoride compounds.

19. A method as in claim 17 wherein said protective dental balm contains an adhesive base material effective for maintaining said active ingredient upon or in close proximity to the surface of said tooth structure.

20. A method as in claim 17 wherein the removal of the protective balm is accomplished by wiping the treated tooth structure with a clean, dry napkin, towel, swab, cloth, paper product or by brushing with toothpaste.
